(19) United States
(12) Patent Application Publication
(10) Pub. No.: US 2022/0075356 A1
(43) Pub. Date: Mar. 10, 2022

(54) POTENCY TESTING

(71) Applicant: Radient Technologies Innovations Inc., Edmonton (CA)

(72) Inventors: Michael Cabigon, Edmonton (CA); Jim Seethram, Edmonton (CA); Steven Splinter, Edmonton (CA); Denis Taschuk, Edmonton (CA)

(21) Appl. No.: 17/238,010

(22) Filed: Apr. 22, 2021

Related U.S. Application Data
(63) Continuation of application No. PCT/IB2019/058950, filed on Oct. 21, 2019.
(60) Provisional application No. 62/749,073, filed on Oct. 22, 2018.

(51) Int. Cl. G05B 19/418; G01N 33/94; A24B 15/16; A24D 1/18

(52) U.S. Cl. CPC G05B 19/41875; G05B 19/4183; A24D 1/18; A24B 15/16; G01N 33/948

(57) ABSTRACT

A system and method for testing the potency of a Cannabis feedstock used in the manufacture of Cannabis-infused foods and beverages are disclosed herein. The system and methods can include at least one sensor capable of detecting cannabinoid compounds. The testing method can include a first test on the incoming Cannabis active ingredients to determine the incoming cannabinoid levels. The results of the first test are used in an algorithm to determine the process steps to incorporate the feedstock into the manufacture of an edible Cannabis product while maintaining a predetermined cannabinoid level in the final product.

### CROSS-REFERENCE TO RELATED APPLICATIONS

The present patent application is a continuation of International Application No. PCT/IB2019/058950 filed Oct. 21, 2019, which claims the priority benefit of U.S. provisional patent application No. 62/749,073 filed Oct. 22, 2018, the disclosures of which are incorporated by reference herein.

### BACKGROUND OF THE INVENTION

### 1. Field of the Disclosure

The present disclosure is generally related to methods and systems for testing the potency of a Cannabis extract used as feedstock in the manufacture of edible Cannabis food products. More specifically the present disclosure relates to using various sensors to monitor the manufacturing process, where the sensor data can be used to adjust and modify the manufacturing process to improve efficiency.

### 2. Description of Related Art

Cannabis is a genus belonging to the family cannabaceae. There are three common species of Cannabis including Cannabis stavia, Cannabis indica, and Cannabis ruderalis. The genus cannabaceae is indigenous to Central Asia and the Indian subcontinent and has a long history of being used for medicinal, therapeutic, and recreational purposes. For example, Cannabis is known to be capable of relieving nausea (such as that accompanying chemotherapy), pain, vomiting, spasticity in multiple sclerosis, and increase hunger in anorexia. The term Cannabis as used herein can refer to a “Cannabis biomass” which can encompass the Cannabis sativa plant and variants thereof, including subspecies sativa, indica and ruderalis, Cannabis cultivars, and Cannabis chemovars (varieties characterised by chemical composition). The term “Cannabis biomass” is to be interpreted accordingly as encompassing plant material derived from one or more Cannabis plants. Such Cannabis biomasses can naturally contain different amounts of the individual cannabinoids.

Each Cannabis biomass contains a unique class of terpeno-phenolic compounds known as cannabinoids, or phytocannabinoids. The principle cannabinoids present in a Cannabis biomass can include delta-9-tetrahydrocannabinolic acid (THCA) and cannabidiolic acid (CBDA). THCA does not include psychoactive properties on it's own, but when decarboxylated THCA becomes delta-9-tetrahydrocannabinol (THC), which is a potent psychoactive cannabinoid. CBDA can be decarboxylated into cannabidiol (CBD), which is a major cannabinoid substituent in hemp Cannabis. CBD is a non-psychoactive cannabinoid and is widely known to have therapeutic potential for a variety of medical conditions including, but not limited to, those described above.

Historical delivery methods of cannabinoids have included combustion (such as smoking) of the dried Cannabis plant material, or biomass. However, smoking can result in adverse effects on a user's respiratory system and is an inefficient delivery mechanism. Common alternative delivery methods, including but not limited to, ingestion, typically require an extraction process to be performed on the Cannabis biomass to remove the desired components. Such ingestible Cannabis items can include, but are not limited to, concentrates, extracts, and Cannabis oils. Often, such ingestible Cannabis items, or Cannabis extracts, are formulated using convenient pharmacologically acceptable diluents, carriers, and/or excipients to produce a composition, which are collectively known as Cannabis derivative products.

A Cannabis edible, also known as a Cannabis-infused food, edible Cannabis product, or simply an “edible,” is a food product which contains one or more cannabinoids, as described above. Although the term “edible” may refer to either a food or a drink, a Cannabis-infused drink may be referred to as a liquid edible or “drinkable.” For the purposes of this disclosure, “food product” can encompass any form of Cannabis edible including liquid edibles. Most edibles contain a significant amount of THC, which can induce a wide range of effects, including relaxation, euphoria, increased appetite, fatigue, and anxiety. THC-dominant edibles are consumed for recreational and medical purposes. In the alternative, some edibles can only contain a negligible amount of THC, and are intended to provide other cannabinoids, most commonly cannabidiol (CBD). Such CBD edibles are primarily used for medical purposes. Foods and beverages made from non-psychoactive Cannabis products are sometimes known as hemp foods.

Food products containing Cannabis extract (edibles) have emerged as a popular and lucrative facet of the legalized Cannabis market for both recreational and medicinal uses. However, there is growing concern about the danger that edibles can pose to children and inexperienced Cannabis consumers, who may easily consume too high of a dose, possibly not even realizing the food product has been infused with a Cannabis extract. Comparing the effects of consuming an edible Cannabis product and smoking Cannabis is extremely difficult because there are large margins of error due to variability in how different people smoke, including the number, duration, and spacing of puffs, the hold time and the volume of the person's lungs all affecting the resulting dosage. With regard to ingesting an edible, the different vehicles in which the cannabinoids are dissolved for oral intake can affect the absorption and bioavailability of the cannabinoids. Furthermore, different people can metabolize the same product at different rates.

In order to provide consistent Cannabis products at scale, many edible Cannabis product manufacturers use an extracted Cannabis product as a feedstock, to provide a more consistent and predictable potency as compared to using the whole plant or flowers of the plant. However, because the dosage of cannabinoids in a food product is highly regulated, there exists a need to automate and regulate the potency of a Cannabis feedstock used in the manufacture of a food product.

### SUMMARY OF THE CLAIMED INVENTION

Examples of the present disclosure provide systems and methods for evaluating the potency of an edible Cannabis product. In particular, a system and method for evaluating the potency, or cannabinoid content, of edible Cannabis products can include a plurality of sensors throughout a production process; the sensors can be operable to visually evaluate the products as they move throughout the process. Additionally, the system can include a sampling device to remove samples of the product at various points throughout the process and perform laboratory analyses to determine an actual cannabinoid content. The information from both the sensors and the lab analyses can be then be used to adjust the manufacturing process in order to ensure the Cannabis content of the final product does not exceed regulated limits.

In addition to verifying the potency of the Cannabis product throughout the manufacturing process, the systems and methods described herein can provide Cannabis product manufacturers a way to ensure consistent products throughout their production process. The systems and methods described herein can be operable to alert a Cannabis product manufacturer when a product is outside the regulated limits and adjust the manufacturing process in order to bring the products back within the proper limits.

### BRIEF DESCRIPTIONS OF THE DRAWINGS

FIG. 1 illustrates an exemplary network environment in which a Cannabis potency testing system may be implemented.

FIG. 2 is a flowchart illustrating an exemplary method for transmitting information using a Cannabis analysis network.

FIG. 3 is a flowchart illustrating an exemplary method for transmitting information using an extractor analysis network.

FIG. 4 is a flowchart illustrating an exemplary method for transmitting information using an extractor network.

FIG. 5 is a flowchart illustrating an exemplary method for evaluating a Cannabis product manufacturing process.

FIG. 6 is a flowchart illustrating an exemplary method for analyzing a Cannabis product manufacturing process.

### DETAILED DESCRIPTION

As described above, the cannabinoids which are orally consumed, such as edibles, can have different absorption rates in different people. Generally, because oral Cannabis doses of are processed by the digestive system and the liver before entering the bloodstream, ingested cannabinoids may be absorbed more slowly, have delayed and lower peak concentrations, and are cleared through the user's system more slowly as compared to an inhalation of the same amount of cannabinoids. Oral administration of cannabinoids generally leads to two concentration peaks, due to enterohepatic circulation. Consuming THC through ingestion results in absorption through the liver and, through metabolic processes, the conversion of a significant proportion of the THC into 11-hydroxy-THC, which is more potent than THC.

Additionally, as discussed above, Cannabis infused edibles may be difficult to identify. For example, one is not usually able to distinguish between regular baked goods and those containing cannabinoids prior to consumption. A Cannabis-infused drink, or a drink which has been infused with cannabinoids, is also difficult to identify prior to consumption. In addition to food and liquid edibles, capsules containing THC or CBD can provide the same effect as food and drink. Such capsules are typically not sold as regulated pharmaceuticals. Cannabis tincture is an alcoholic extract of Cannabis; because Cannabis resins are soluble in alcohol, an effective way of adding them into dishes is through the use of cooking brandy or rum infused has been with cannabinoids. Due to the vast number of ways to introduce cannabinoids into food products or food related production processes, there is a need for a system or method operable to control the potency of the Cannabis active ingredient in the final edible food product. Furthermore, due to the potential for overdose, as described above, many governments have instituted regulations dictating the amount of cannabinoids which can be added to edibles.

The systems and methods provided in the present disclosure allow for the use of currently available sensors and future sensors to assist the production of Cannabis infused food products. The present disclosure additionally focuses on inspection of the Cannabis active ingredient product, for example Cannabis extract, as well as inspection of other parts of the Cannabis infused food production process. The methods and systems described herein can be used to adjust or modify the production processes to improve efficiency and consistency. As such, the systems and methods described herein can directly improve the repeatability and reliability of the Cannabis infused food production process. What is provided herein is a system and method for ensuring consistent and repeatable Cannabis infused edibles based upon extracts received from various suppliers and production runs of the Cannabis active ingredients. In at least one example, the consistency is determined based on a potency analysis of the cannabinoids present in the final Cannabis edible product.

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

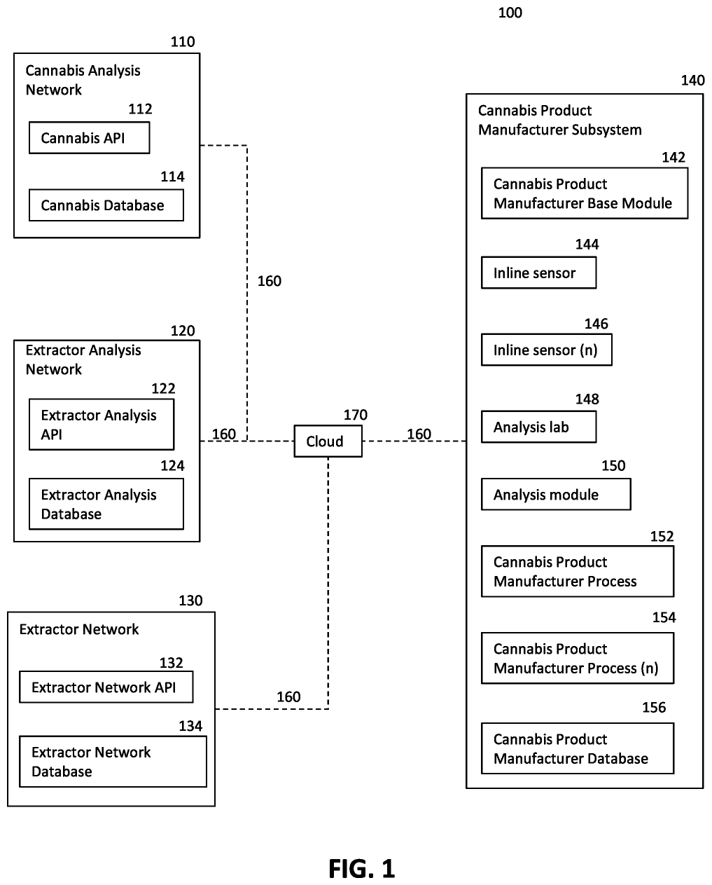

FIG. 1 illustrates an exemplary network environment in which a Cannabis potency testing system may be implemented. Such system 100 may be provided for testing Cannabis potency for a Cannabis-infused food or beverage manufacturer. Specifically, the system 100 of FIG. 1 can include at least a Cannabis analysis network 110, an extractor analysis network 120, an extractor network 130, and a Cannabis product manufacturer subsystem 140. The Cannabis analysis network 110 which can be operable to allow analytical labs which can test Cannabis products for various properties, such as potency, and is operable to make the data centrally available via a Cannabis API 112. The analytical labs can be operable to provide analysis services for legally-authorized and licensed growers, extractors, distributors, producers, and licensed regulators of Cannabis. Such Cannabis analysis labs can focus on the testing of Cannabis biomass, Cannabis extracts, and Cannabis-infused products to determine the potency and safety of products containing active cannabinoids. The Cannabis analytical labs can be operable to provide accurate reporting of cannabinoid content and potency, including but not limited to, THCA, THC, CBDA, CBD and other chemical and physical properties including, but not limited to, terpene profiles, moisture content, microbial content, heavy metals content, and pesticide content. In at least one example, the Cannabis analytical labs can additionally provide an analysis of the genetics of the Cannabis (including Cannabis type and cultivar), Cannabis physiology, and Cannabis biochemistry.

The Cannabis analysis network 110 can also include a Cannabis database 114 stored thereon. The Cannabis database 114 can be operable to store a collection of information which can be organized so that the information can be easily accessed, managed and updated. In at least one example, the Cannabis database 114 can be operable to process workloads in order to create and update themselves, querying the data stored on the database, and running applications against it. The Cannabis analysis network 110 can further include a Cannabis API 112 which can be an application programming interface (API) having stored thereon a set of subroutine definitions, communication protocols, and tools for building software. In general terms, the Cannabis API 112 can include a set of clearly defined methods of communication between various components. A good API can be operable to ease in the development of a computer program by providing all the building blocks, which are then put together by the programmer. An API can be designed for a web-based system, operating system, database system, computer hardware, or software library. An API specification can take many forms, but often includes specifications for routines, data structures, object classes, variables, or remote calls. In at least one example, the API can be a POSIX, a Windows API, and an ASPI. Documentation for the API can usually be provided to facilitate usage and implementation. The Cannabis API 112 can be operable to communicate with the extractor analysis network 120, the extractor network 130, and the Cannabis product manufacturer subsystem 140 via the communication network 160 via a cloud 170.

The extractor analysis network 120, which represents a plurality of extraction companies and provides an extractor analysis database operable to centralize and store the extractors analysis data for others to easily access. The extractor analysis network 120 can be used for extraction data by allowing extractors to store their analysis in an extractor analysis database 124 or to download data from the extractor analysis network 120. The extractor analysis network 120 can be accessed by the plurality of extractors using an extractor analysis API 122. The extractor analysis API 122 can be an API as described above. In at least one example, manufacturers of Cannabis infused food products can receive extraction data, such as that stored on the extractor analysis database 124, from their supplier/extractor. In an alternative example, manufactures of Cannabis infused food products can obtain extraction data directly from the extractor analysis network 120, if the extractor posts such data. Extractors can be encouraged to upload extraction data relating to their Cannabis extracts onto the extractor analysis network 120, as it can be used to help the value chain learn important Cannabis data, and provides Cannabis product manufacturer subsystem 140 with centralized access to current and past extraction data from a given extractor. Furthermore, in at least one example, the extractor analysis network 120 can be searchable, which can be helpful to the extractors as a marketing approach.

The extractor network 130 can represent extraction companies which can be operable to manufacture Cannabis extracts and Cannabis concentrates from a Cannabis biomass. Cannabis extracts may be obtained from Cannabis biomass by any number of methods including, but not limited to, supercritical fluid extraction, solvent extraction, and microwave-assisted extraction. The extractors are able to send and receive extractor data from the extractor network 130 to an extractor network database 132. In addition, the Cannabis product manufacturer subsystem 140 can access the extraction data from the extractor network database 134, when needed. The extractor network database 132 can be accessed via an extractor network API 132. The extractor network API 132 can be an API, as described above, and can be used to upload and download information from the extractor network database 134. The extractor network database 134 can be a collection of information organized such that it can be easily accessed, managed and updated via the extractor network API 132. In at least one example, the extractor network database 134 can process workloads to create and update themselves, querying the data they contain and running applications against it.

The databases on each of the Cannabis analysis network 110, the extractor analysis network 120, and the extractor network 130 can be accessed via the communication network 160. The communication network 160 may be a wired and/or a wireless network. The communication network 160, if wireless, may be implemented using communication techniques such as Visible Light Communication (VLC), Worldwide Interoperability for Microwave Access (WiMAX), Long Term Evolution (LTE), Wireless Local Area Network (WLAN), Infrared (IR) communication, Public Switched Telephone Network (PSTN), Radio waves, and other communication techniques known in the art. The communication network 160 may allow ubiquitous access to shared pools of configurable system resources and higher-level services that can be rapidly provisioned with minimal management effort, often over Internet and relies on sharing of resources to achieve coherence and economies of scale, like a public utility, while third-party clouds 170 enable organizations to focus on their core businesses instead of expending resources on computer infrastructure and maintenance.

The system 100 can further include a Cannabis product manufacturer subsystem 140 which can represent any large or small edible or beverage company that is creating Cannabis infused food products. The Cannabis product manufacturer subsystem 140 can include a Cannabis product manufacturer base module 142 which can be a software program operable to manage all the interactions of the Cannabis infused product manufacturer subsystem 140, from reading all the sensors, inputting and extracting data from the analysis module, extracting Cannabis product manufacturer process data, controlling the Cannabis product manufacturer process, and storing and analyzing data in the Cannabis product manufacturer database 156. Specifically, the Cannabis product manufacturer subsystem 140 can include inline sensor 144, which can be an inline sensor used by the Cannabis food manufacturer to monitor the processes. The inline sensor 144 can include, but is not limited to, a Cannabis sensor (such as that described at https://www.mydxlife.com/canna-sensor/) which can allow the manufacturer to test for Cannabis potency (% THC) on site and within a matter of minutes; a Cannabis sensor operable to detect other cannabinoids (including, but not limited to, CBD and cannabinol (CBN)) as well as several terpenes; and a Cannabis sensor operable to provide a comprehensive chemical analysis. Other sensors compatible with the systems and methods disclosed herein can include, but are not limited to, that described in “Absorptive stripping voltammetry for Cannabis detection” (https://www.ncbi.nlm.nih.gov/pmc/articles/PMC4493815/) which can be operable to measure delta-9-tetrahydrocannabinol (THC). In at least one example, additional sensors can be located throughout the manufacturing process that are operable to detect the impact on Cannabis active ingredients including, but not limited to, temperature sensors, pressure sensors, humidity sensors, and the like. Factors such as temperature, pressure and humidity can detect the impact of the process on the sensors. In addition to the inline sensor 144, the Cannabis product manufacturer subsystem 140 can further include various other inline sensors (n) 146. The inline sensors (n) 146 can be any of the sensors described above.

The Cannabis product manufacturer subsystem 140 can further include a Cannabis analysis lab 148 which can be either internal or external to the Cannabis product manufacturer. The analysis lab 148 can be operable to determine the potency (cannabinoid content) and chemical profile of samples which are received from Cannabis product manufacturers. Additionally, the analysis lab 148 can be operable to insure the products are free from contaminants such as mold, microbial contamination, heavy metals, pesticides, and any other contaminant. In at least one example, the Cannabis analysis lab 148 can be operable to analyze samples from the manufacturing process at various time intervals, some historical, and some at critical gates The data obtained from the analysis at various time intervals can be used to improve various parts of the process. The Cannabis product manufacturer subsystem 140 can further include a sampling device, not shown, operable to take Cannabis food product samples at various steps throughout the production process. In at least one example, the sampling device can be operable to obtain samples from the production process before and after mixing, before and after heat treatments, or before and after any other process step where changes can occur in the cannabinoids used. As such, the Cannabis analysis lab 148 can be operable to provide quality control checks throughout the process including, but not limited to, a percentage of the sample volume, or potency and chemical composition versus impact (including the taste, levels of sugar, and other factors effecting final Cannabis product quality) of the food sample. The Cannabis product manufacturer subsystem 140 can also include a Cannabis analysis module 150 which can be a software module that is operable to receive data from various sensors, data from the Cannabis analysis lab 148, and any other data transmitted to the analysis module 150 to control the use of a Cannabis concentrate as an additive in a food product. In at least one example, the analysis module 150 can be operable to determine the amount of Cannabis extract (dosage) to add to a specific desired Cannabis product based on historical data. The Cannabis analysis module 150 can also create and establish reports relating to the quality and control of the Cannabis concentrate throughout the production process.

The Cannabis product manufacturer subsystem 140 can also include a Cannabis product manufacturer process 152. The Cannabis product manufacturer process 152 can include elements such as mixing, shredding, extruding food processes, vibratory conveyors, a bucket, distribution systems, hopper-feeders, in-process storage systems, mix/blend systems, shuffle flows, hydrostatic steam blancher, hydro chiller, wash system, and combinations thereof. The Cannabis product manufacturer subsystem 140 can include any number of Cannabis product manufacturer processes (n) 154. The Cannabis product manufacturer processes (n) 154 can include any of the process elements described above. The additional Cannabis product manufacturer processes (n) 154 can be operable to interact with the Cannabis product manufacturer process 152.

Finally, the information gathered via the inline sensor 144, inline sensors (n) 146, analysis lab 148, analysis module 150, processes 152, processes (n) 154, and the sampling device can be stored on a Cannabis product manufacturer database 156. The Cannabis product manufacturer database 160 can be a collection of information that is organized such that it can be easily accessed, managed and updated. In at least one example, the Cannabis product manufacturer database 156 can process workloads to create and update themselves, querying the data they contain and running applications against it.

While the present disclosure generally refers to Cannabis edibles as being produced by Cannabis food or beverage manufacturers using Cannabis extracts or concentrates manufactured by Cannabis extractors, it should be recognized that in some cases, the Cannabis product manufacturer and the Cannabis extractor can be the same entity.

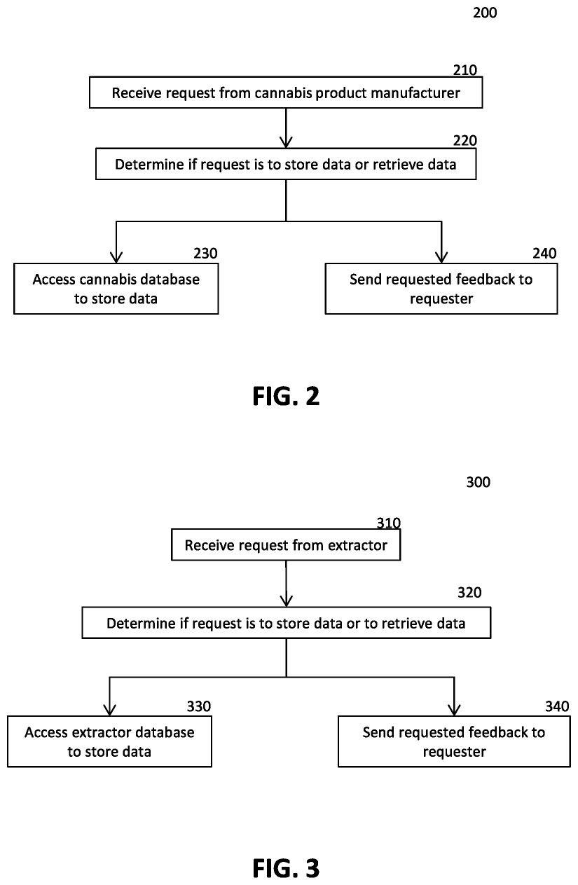

FIG. 2 is a flowchart illustrating an exemplary method for transmitting information using a Cannabis analysis network. Such method 200 may be provided for transmitting data to and from a Cannabis product manufacturer. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

Specifically, the method 200 can begin at block 210 where the Cannabis analysis network 110 receives a request from Cannabis product manufacturers. At block 220, the Cannabis analysis network can determine if the request is a request to store data from an extractor on the Cannabis database 114, or a request to retrieve data from the Cannabis database 114 to send to the Cannabis product manufacturer. If the Cannabis analysis network 110 determines that the request is a request from an extractor to store data, the method 200 can proceed to block 230. At block 230, the Cannabis analysis network 110 can allow access to the Cannabis database 114 via the communication network 160 and store the data on the Cannabis database 114. In the alternative, if the Cannabis analysis network 110 determines the request is from a Cannabis product manufacturer to send data the method 200 can proceed to block 240. At block 240, the Cannabis analysis network 110 can access the Cannabis database 114 to retrieve the desired data and send the requested data back the requester via the communication network 160.

FIG. 3 is a flowchart illustrating an exemplary method for transmitting information using an extractors analysis network. Such method 300 may be provided for transmitting data to and from an extractor analysis network 120. The method 300 of FIG. 3 can begin at block 310, where a request is received at the extractor analysis network 120 from one or more extractors. At block 320, the extractor analysis network 120 can determine whether the request is a request to store data from an extractor on the extractor analysis database 124, or a request to retrieve data from the extractor analysis database 124 to send back to the extractors. If the extractor analysis network 120 determines that the request is to store information on the extractor database 124, the method 300 can proceed to block 330. At block 330, the extractor analysis network 124 can allow access to the extractor analysis database 124 via the communication network such that the data can be stored on the extractor analysis database 124. In the alternative, if the request is determined to be one to retrieve data, the method 300 can proceed to block 340. At block 340, the extractor analysis network 120 can retrieve the requested data from the extractor analysis database 124, and send the desired data to the requester via the communication network 160.

An exemplary entry from the extractor analysis database 124 is provided in Table 1, below. It should be understood that the information provided in Table 1 is merely illustrative for the purposes of this disclosure and is not intended to be limiting.

TABLE 1

| Lot | | Date of | THC:CBD | Residual | Extract | Extraction |
|---|---|---|---|---|---|---|
| Number | Extractor | Extraction | ratio | Solvent (%) | Quality | Efficiency |
| 151 | Company | Aug. 28, 2017 | 1:01 | 0 | High | High |
| | ABC | | | | | |
| 158 | Company | Aug. 31, 2017 | 1.5:1 | 0 | High | Medium |
| | ABC | | | | | |
| 161 | Company | Sep. 3, 2017 | 1:01 | 0.01 | High | Medium |
| | DEF | | | | | |
| 166 | Company | Sep. 10, 2017 | 1.2:1 | 0.2 | High | High |
| | ABC | | | | | |
| . . . | . . . | . . . | . . . | . . . | . . . | . . . |
| . . . | . . . | . . . | . . . | . . . | . . . | . . . |
| 201 | Company | Sep. 29, 2015 | 1.3:1 | 0 | Medium | High |
| | ABC | | | | | |

The extractor analysis database 124 can be operable to store data including, but not limited to, Lot Number, extractor (extraction company), date of extraction, cannabinoid potency (such as percentage THC, percentage CBD), residual solvent (contaminant) percentage, extract quality, cannabinoid extraction efficiency. As shown, the data stored in the extractor analysis database 124 can be useful to extractors in determining the efficiency of their extracted product as compared to previous extractions or as compared to other extractors. The information can be stored by lot number, as illustrated in Table 1, to allow for easier tracking.

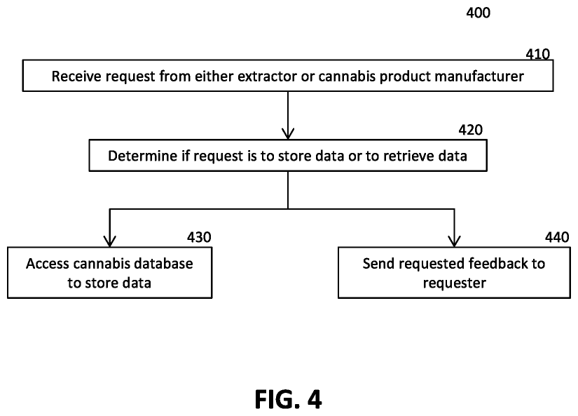

FIG. 4 is a flowchart illustrating an exemplary method for transmitting information using an extractor network. The method 400 may be provided for transmitting information to and from the extractor network 130. The method 400 can begin at block 410 where the extractor network 130 can receive a request from either an extractor or a Cannabis product manufacturer. At block 420, the extractor network 130 can determine whether the request is a request to store data from an extractor to the extractor network database 134, or a request to retrieve data from the extractor network database 134 to send to the Cannabis product manufacturer. If the extractor network 130 determines that the request is a request from an extractor to store data, the method 400 can proceed to block 430. At block 430, the extractor network 130 can allow access to the extractor network database 134, via the communication network 160, to store data on the extractor network database 134. In the alternative, if the extractor network 130 determines that the request is a request to retrieve information received from a Cannabis product manufacturer, the method 400 can proceed to block 440. At block 440, the extract network 134 can retrieve the requested data from the extractor network database 134 and send the requested data to the Cannabis product manufacturer via the communication network 160.

An exemplary entry from the extractor network database 134 is provided as Table 2, below. It should be understood that the information provided in Table 2 is merely illustrative for the purposes of this disclosure and is not intended to be limiting.

TABLE 2

| Lot | | Date of | Potency % | | Extract | Extraction |
|---|---|---|---|---|---|---|
| Number | Extractor | Extraction | (THC, CBD) | Solvent | Quality | Efficiency |
| 151 | Company | Aug. 28, 2017 | High | Ethanol | High | High |
| | ABC | | | | | |
| 158 | Company | Aug. 31, 2017 | High | Ethanol | High | Medium |
| | ABC | | | | | |
| 161 | Company | Sep. 3, 2017 | High | Ethanol | High | Medium |
| | DEF | | | | | |
| 166 | Company | Sep. 10, 2017 | High | Ethanol | High | High |
| | ABC | | | | | |
| . . . | . . . | . . . | . . . | . . . | . . . | . . . |
| . . . | . . . | . . . | . . . | . . . | . . . | . . . |
| 201 | Company | Sep. 29, 2015 | High | Ethanol | Medium | High |
| | ABC | | | | | |

The extractor network database 134 can be operable to store information including, but not limited to, Cannabis biomass Lot Number, Extractor (extraction company), date of extraction, Cannabis biomass potency, solvent used, Cannabis extract potency; extract quality, and cannabinoid extraction efficiency. As described with respect to FIG. 4, the exemplary extractor network database provided as Table 2 can include information valuable to both extractors and Cannabis product manufacturers. In at least one example, a Cannabis product manufacturer can use the information provided by the extractor network database 134 to determine the company that produced the Cannabis extract they are using in their manufacturing process as well as the potency of the Cannabis extract they are using.

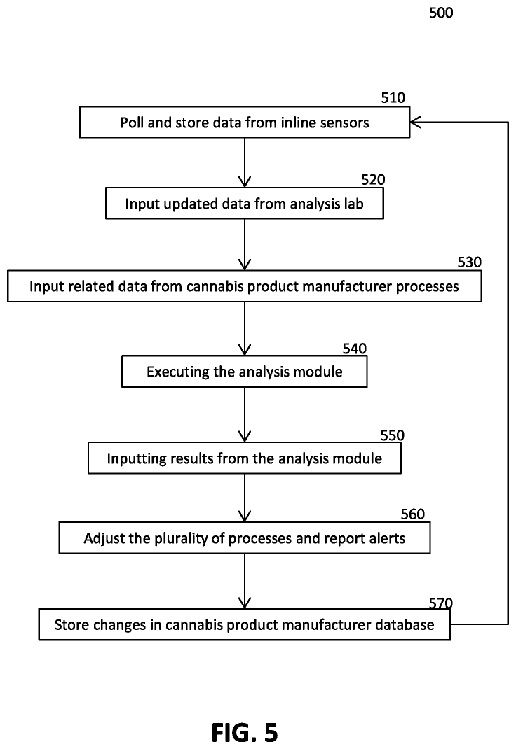

FIG. 5 is a flowchart illustrating an exemplary method for evaluating a Cannabis product manufacturing process. The method 500 can being at block 510 where the Cannabis product manufacturer base module 142 polls data from each of the inline sensors 144,146 and stores the data in the Cannabis product manufacturer database 156. As described above, the data obtained from the inline sensors 144,146 can be a Cannabis sensor. At block 520, any updated data received from the Cannabis analysis lab 148 is input into the Cannabis product manufacturer database 156. As described above, the analysis lab 148 can provide cannabinoid content (potency) information relating to various points throughout the production process. At block 530, any related data from the Cannabis product manufacturer processes 152,154 is input and stored in the Cannabis product manufacturer database 156.

At block 540, the Cannabis product manufacturer 142 can execute the analysis module 150. The analysis module can evaluate the Cannabis product in production in order to determine the potency of the cannabinoids contained within the products. At block 550, the Cannabis product manufacturer base module 142 can input the results from the analysis module 150 into the Cannabis product manufacturer database 156.

At block 560, the Cannabis product manufacturer base module 142 can adjust one or more of the Cannabis product manufacturer processes 152,154 based on the analysis. For example, the Cannabis product manufacturer base module 142 can be operable to make the necessary changes to one or more of the processes 152,154 in order to produce a more ideal Cannabis product based on the tests performed on products sampled at various points throughout production. The Cannabis product manufacturer base module 142 can also be operable to issue one or more alerts related to the changes made to the processes 152,154. The one or more alerts can be operable to inform the Cannabis product manufacturer of the changes made to the processes 152,154. Finally, at block 570, each of the changes made to the processes 152,154 can be stored in the Cannabis product manufacturer database 156. The method 500 can be repeated as necessary to provide Cannabis products having consistent potency.

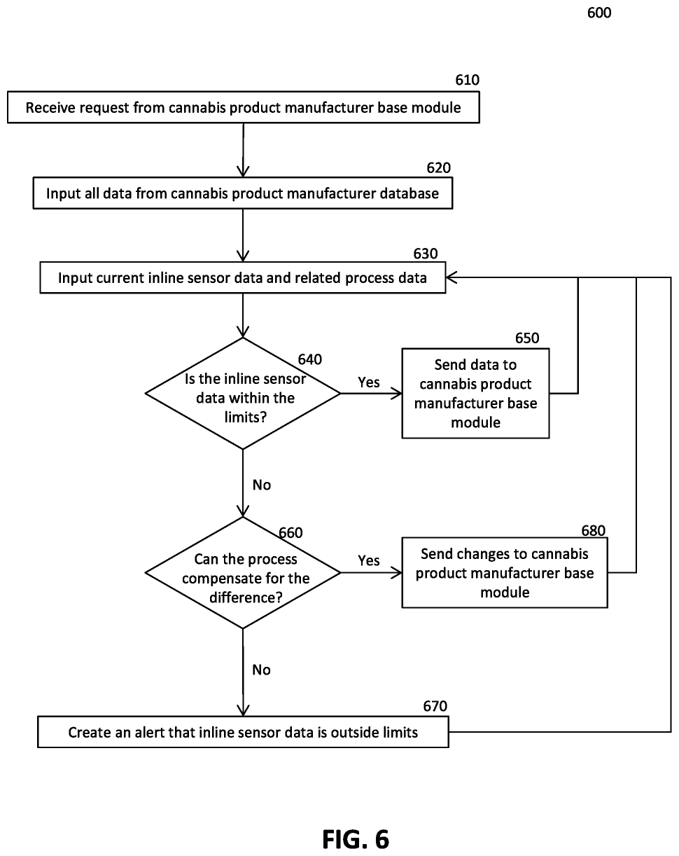

FIG. 6 is a flowchart illustrating an exemplary method for analyzing a Cannabis product manufacturing process. Such method 600 may result from execution of the analysis module 150. Specifically, FIG. 6 illustrates a method 600 for evaluating the efficiency of a Cannabis product production process. The method 600 can begin at block 610 where the Cannabis product manufacturer analysis module 150 receives a request from the Cannabis product manufacturer base module 142. At block 620, all the data received from the Cannabis product manufacturer database 156 is input into the analysis module 150. At block 630, current inline sensor 144,146 data relating to a single sensor can be input into the analysis module 150. The inline sensor 144,146 data can include a correlation to Cannabis product manufacturer process 152,154 data.

At block 640, the analysis module 150 determines whether the inline sensor 144,146 data is within a set of predetermined limits. For example, the predetermine limits can include, but are not limited to, THC concentration (such as the THC concentration is higher or lower than the limit). If the analysis module 150 determines that the inline sensor 144,146 data is within the predetermined limits, the method 600 can proceed to block 650. At block 650, the data is transmitted to the Cannabis product manufacturer base module. The method 600 can proceed to block 630, where the process is repeated for each of the inline sensors 144,146 within the production system.

In the alternative, if the method 600 determines that the inline sensor 144,146 data is not within the limits at block 640, then the method 600 proceeds to block 660. At block 660, the analysis module 150 evaluates whether or not one or more of the processes 152,154 can be adjusted to bring the inline sensor 144,146 data within the predetermined limits. If the analysis module 150 determines that one or more of the processes 152,154 can be adjusted to compensate for the error (such as mixing a smaller volume of the food product), the method 600 can proceed to block 680. In at least one example, the adjustment can be to change the point within the manufacturing process where the Cannabis extract is added to the Cannabis product. In at least one example, the adjustment can be operable to increase the uniform distribution of the Cannabis extract throughout the Cannabis product being produced. At block 680, the analysis module 150 can send the process 152,154 changes to the Cannabis product manufacturer base module 142. In at least one example, an algorithm may be executed in the analysis module to determine one or more of a (1) specific dilution ratio for the Cannabis active ingredient product to a first stage mixing, a (2) dosing scheme as to where in the food production the Cannabis active ingredient product is added in terms of amount, and (3) an amount of edible Cannabis infused food product that may result for a given ratio and amount of active ingredients. Various algorithms known in the field are can assist the food manufacturers. The changes sent to the Cannabis product manufacturer base module 142 can include information relating to the particular process 152,154 which have been affected. The method 600 can then proceed to block 630, where the process is repeated for each of the inline sensors 144,146.

Finally, if the analysis module 150 determines at block 660 that the none of the processes 152,154 can be adjusted to compensate for the inline sensor 144,146 data being outside the predetermined limit, the method 600 proceeds to block 670. At block 670, the analysis module 150 generates an alert to send to the Cannabis product manufacturer base module 142. The alert can indicate that one or more of the inline sensors 144,146 are outside the predetermined limits and that none of the processes 152,154 can be adjusted in order to compensate for the sensor data. The method 600 can then proceed to block 630, where the process is repeated for each of the inline sensors 144,146.

An exemplary entry from the Cannabis product manufacturer database 156 is provided as Table 3, below. It should be understood that the information provided in Table 3 is merely illustrative for the purposes of this disclosure and is not intended to be limiting.

TABLE 3

| | | | | | | Sensor | Sensor | | | | | | | |
|---|---|---|---|---|---|---|---|---|---|---|---|---|---|---|
| Product | | | Lot | Sensor | Sensor | . . . | . . . | Sensor | Sensor | Process | Process | Process | Process | Analysis |
| Type | Date | Time | No. | 1 Data | 1 Limit | Data | Limit | n Data | n Limit | 1 Data | 1 Limit | n Data | n Limit | Lab Ref. |
| Candy | Aug. 28, | 8:00 | 151 | 15% | 13% | 35% | 20% | 11% | 10% | 32 | 30 | 16 | 15 | R311 |
| | 2017 | | | | | | | | | | | | | |
| Candy | Aug. 28, | 9:00 | 151 | 13% | 13% | 35% | 20% | 12% | 10% | 32 | 30 | 16 | 15 | R312 |
| | 2018 | | | | | | | | | | | | | |
| Candy | Sep. 1, | 10:00 | 151 | 13% | 13% | 34% | 20% | 9% | 10% | 32 | 30 | 16 | 15 | R313 |
| | 2017 | | | | | | | | | | | | | |
| . . . | . . . | . . . | . . . | . . . | . . . | . . . | . . . | . . . | . . . | . . . | . . . | . . . | . . . | . . . |
| . . . | . . . | . . . | . . . | . . . | . . . | . . . | . . . | . . . | . . . | . . . | . . . | . . . | . . . | . . . |
| Candy | Sep. 4, | 8:00 | 152 | 11% | 13% | 21% | 20% | 11% | 10% | 28 | 30 | 12 | 15 | R411 |
| | 2017 | | | | | | | | | | | | | |
| Candy | Sep. 4, | 9:00 | 152 | 13% | 13% | 19% | 20% | 11% | 10% | 28 | 30 | 12 | 15 | R412 |
| | 2017 | | | | | | | | | | | | | |
| Brownie | Sep. 6, | 8:00 | 161 | 13% | 13% | 19% | 20% | 11% | 10% | 32 | 30 | 16 | 15 | R413 |
| | 2017 | | | | | | | | | | | | | |
| Brownie | Sep. 13, | 9:00 | 161 | 16% | 13% | 21% | 20% | 12% | 10% | 32 | 30 | 16 | 15 | R141 |
| | 2017 | | | | | | | | | | | | | |
| Brownie | Sep. 15, | 10:00 | 161 | 16% | 13% | 21% | 20% | 8% | 10% | 32 | 30 | 16 | 15 | R415 |
| | 2017 | | | | | | | | | | | | | |
| . . . | . . . | . . . | . . . | . . . | . . . | . . . | . . . | . . . | . . . | . . . | . . . | . . . | . . . | . . . |
| . . . | . . . | . . . | . . . | . . . | . . . | . . . | . . . | . . . | . . . | . . . | . . . | . . . | . . . | . . . |
| Brownie | Sep. 20, | 8:00 | 166 | 15% | 13% | 35% | 20% | 11% | 10% | 32 | 30 | 16 | 15 | R420 |
| | 2016 | | | | | | | | | | | | | |
| Brownie | Sep. 21, | 9:00 | 166 | 15% | 13% | 35% | 20% | 11% | 10% | 32 | 30 | 16 | 15 | R421 |
| | 2017 | | | | | | | | | | | | | |

The Cannabis product manufacturer database 156 can be operable to store information including, but not limited to, food product type, date, time, lot number, inline sensor 1 data, inline sensor 1 limit, inline sensor . . . data, inline sensor . . . limit, inline sensor n data, inline sensor n limit, Cannabis product manufacturer process data, Cannabis product manufacturer process limit, Cannabis product manufacturer process n data, Cannabis product manufacturer process n limit, and Cannabis analysis lab reference. For each record in the Cannabis product manufacturer database 156, a historical final run Cannabis analysis lab can be run. The data obtained from the analysis lab can then be stored in the Cannabis product manufacturer database 156 and correlated to a specific reference file. In at least one example, the data stored in the Cannabis product manufacturer base module 156 can be used to determine the appropriate dosage or Cannabis extract for a manufacturer to include in a specific food product, based on the historical data.

The foregoing detailed description of the technology has been presented for purposes of illustration and description. It is not intended to be exhaustive or to limit the technology to the precise form disclosed. Many modifications and variations are possible in light of the above teaching. The described embodiments were chosen in order to best explain the principles of the technology, its practical application, and to enable others skilled in the art to utilize the technology in various embodiments and with various modifications as are suited to the particular use contemplated. It is intended that the scope of the technology be defined by the claims.

What is claimed is:

1. A method for evaluating the potency of an edible Cannabis product, the method comprising:
receiving, via a communication network, data sent from one or more inline sensors of a manufacturing system that is producing an edible Cannabis product;
inputting data into an analysis module, the inputted data including data from an analysis lab and data relating to one or more Cannabis product manufacturer processes;
executing the analysis module to determine a potency of the edible Cannabis product based on the data from the one or more inline sensors and the inputted data; and
identifying if the determined potency of the edible Cannabis product is within a predetermined limit.

2. The method of claim 1, wherein the one or more inline sensors are selected from the group comprising a Cannabis sensor, a temperature sensor, a humid sensor, and a pressure sensor.

3. The method of claim 1, wherein the data from the one or more inline sensors indicates one or more of a Cannabis potency, a Cannabis concentration, a terpene profile, and a comprehensive analysis of the edible Cannabis product.

4. The method of claim 1, wherein the one or more inline sensors are spaced at different locations throughout the manufacturing system.

5. The method of claim 4, wherein the determined potency is specific to a first inline sensor of the one or more inline sensors spaced throughout the manufacturing system, and further comprising:
determining whether the first inline sensor is outside the predetermined limits; and when the first inline sensor is outside the predetermined limits:
adjusting at least one of the one or more Cannabis production manufacturer processes to bring the inline sensor data within the predetermined limits.

6. The method of claim 5, wherein the predetermined limit is a delta-9-tetrahydrocannabinol (THC) content.

7. The method of claim 1, wherein the predetermined limit corresponds to government regulations inputted into the analysis module.

8. A system for evaluating the potency of an edible Cannabis product, the system comprising:
a communication network interface that receives, via a communication network, data sent from one or more inline sensors of a manufacturing system that is producing an edible Cannabis product;
an analysis module that received inputted data, the inputted data including data from an analysis lab and data relating to one or more Cannabis product manufacturer processes; and
a processor that executes the analysis module to:
determine a potency of the edible Cannabis product based on the data from the one or more inline sensors and the inputted data; and
identify if the determined potency of the edible Cannabis product is within a predetermined limit.

9. The system of claim 8, wherein the one or more inline sensors are selected from the group comprising a Cannabis sensor, a temperature sensor, a humid sensor, and a pressure sensor.

10. The system of claim 8, wherein the data from the one or more inline sensors indicates one or more of a Cannabis potency, a Cannabis concentration, a terpene profile, and a comprehensive analysis of the edible Cannabis product.

11. The system of claim 8, wherein the one or more inline sensors are spaced at different locations throughout the manufacturing system.

12. The system of claim 11, wherein the determined potency is specific to a first inline sensor of the one or more inline sensors spaced throughout the manufacturing system, and wherein the processor executes further instructions to:
determine whether the first inline sensor is outside the predetermined limits; and when the first inline sensor is outside the predetermined limits:
adjust at least one of the one or more Cannabis production manufacturer processes to bring the first inline sensor data within the predetermined limits.

13. The system of claim 12, wherein the predetermined limit is a delta-9-tetrahydrocannabinol (THC) content.

14. The system of claim 8, wherein the communication network interface further receives data from an extractor network database stored on an extractor network server.

15. The system of claim 8, wherein the communication network interface further receives data from a Cannabis database stored on a Cannabis analysis network server.

16. The system of claim 15, wherein the data received from the Cannabis database includes a cannabinoid concentration of the Cannabis extract.

17. A non-transitory, computer-readable storage medium, having embodied thereon a program executable by a processor to perform a method for evaluating potency of an edible Cannabis product, the method comprising:
receiving, via a communications network, data sent from one or more inline sensors of a manufacturing system that is producing an edible Cannabis product;
inputting data into an analysis module, the inputted data including data from an analysis lab and data relating to one or more Cannabis product manufacturer processes;
executing the analysis module to determine a potency of the edible Cannabis product based on the data from the one or more inline sensors and the inputted data; and
identifying if the determined potency of the edible Cannabis product is within a predetermined limit.
